(19) United States
(12) Patent Application Publication
(10) Pub. No.: US 2021/0196402 A1
(43) Pub. Date: Jul. 1, 2021

(54) SYSTEMS AND METHODS FOR SUBJECT POSITIONING AND IMAGE-GUIDED SURGERY

(71) Applicant: SHANGHAI UNITED IMAGING HEALTHCARE CO., LTD., Shanghai (CN)

(72) Inventors: Hui YIN, Shanghai (CN); Yecheng HAN, Shanghai (CN); Yong E, Shanghai (CN); Wanli TENG, Shanghai (CN); Yajun LIU, Shanghai (CN); Wei TIAN, Shanghai (CN)

(73) Assignee: SHANGHAI UNITED IMAGING HEALTHCARE CO., LTD., Shanghai (CN)

(21) Appl. No.: 17/190,375

(22) Filed: Mar. 2, 2021

Related U.S. Application Data
(63) Continuation of application No. PCT/CN2019/123838, filed on Dec. 7, 2019.

(30) Foreign Application Priority Data
Dec. 7, 2018 (CN) 201822057380.9
Dec. 29, 2018 (CN) 201811641080.3

(51) Int. Cl. A61B 34/20; A61B 90/50; A61B 17/3211; A61B 17/34; A61B 5/00

(52) U.S. Cl. CPC A61B 34/20; A61B 90/50; A61B 17/3211; A61B 2090/376; A61B 5/7405; A61B 5/745; A61B 5/70; A61B 17/34

(57) ABSTRACT

The present disclosure is related to a system. The system may include a medical device, a medical device, a storage device, a processing device, and an interaction device. The medical device may be configured to perform a medical procedure on a subject or a portion thereof. The storage device may be configured to store subject procedure information relating to the medical procedure on the subject. The processing device may be configured to communicate with the medical device, the storage device, and the interaction device. The interaction device may be configured to communicate to the subject at least a portion of the subject procedure information during the medical procedure.

### CROSS-REFERENCE TO RELATED APPLICATIONS

This application is a continuation of International Patent Application No. PCT/CN2019/123838, filed on Dec. 7, 2019, which claims priority of Chinese Patent Application No. 201811641080.3, filed on Dec. 29, 2018, and Chinese Patent Application No. 201822057380.9, filed on Dec. 7, 2018, the contents of each of which are hereby incorporated by reference.

### TECHNICAL HELD

This disclosure generally relates to an imaging system, and more particularly, relates to systems and methods for subject positioning in the imaging system and/or image-guided surgery.

### BACKGROUND

Medical imaging systems have been widely used in clinical examinations, and medical diagnosis and treatment in recent years. When an imaging device (e.g., an X-ray imaging device) is used to perform a scan, a subject needs to be positioned, holding a specific posture so that a target portion of the subject can be imaged and/or treated effectively. For a simple preset posture, a user can help the subject adjust his/her posture by talking to the subject. However, for a complex preset posture, the user needs to instruct the subject personally. The positioning process is generally complicated, time consuming, and/or has low accuracy, which may influence the efficiency of an imaging and/or treatment process. Therefore, it is desirable to provide systems and methods for facilitating the positioning of a subject in an imaging and/or treatment process.

As another example, in a medical operation (e.g., a puncture surgery, a minimally invasive surgery), the user generally needs to look back and forth between a subject and one or more monitors displaying anatomical information associated with the subject for guidance in operation. Usually, the user may perform the operation with the assistance of a puncture positioning device and an imaging device, which may be inconvenient for the user and may cause the user to be exposed to harmful radiation. Therefore, it is desirable to provide systems and methods for facilitating image-guided surgery.

### SUMMARY

According to an aspect of the present disclosure, a system is provided. The system may include an imaging device, a storage device, a processing device, and interaction device. The imaging device may be configured to generate image data by imaging a subject or a portion thereof. The storage device may be configured to store information regarding a plurality of preset postures. The processing device may be configured to communicate with the imaging device, the storage device, and an interaction device. The interaction device, in communication with the storage device, may be configured to provide to the subject information regarding at least one preset posture of the plurality of preset postures.

In some embodiments, the interaction device may include at least one of an optical device, a projection device, and an audio device.

In some embodiments, the interaction device may include a holographic projector configured to project a first image of the at least one preset posture of the plurality of preset postures.

In some embodiments, the holographic projector may be movable.

In some embodiments, the system may include a movable base configured to carry the holographic projector to move.

In some embodiments, the system may include a control device, in communication with the interaction device, configured to control the interaction device.

In some embodiments, the processing device may further be configured to determine a display position of the information regarding the at least one preset posture based on a position of the imaging device.

In some embodiments, the system may include an image capture device configured to capture a second image representing an actual posture of the subject when the subject is positioned within the imaging device.

In some embodiments, the processing device may further be configured to determine a difference between the actual posture of the subject and the at least one preset posture of the subject. The processing device may further be configured to determine whether the difference is below a threshold.

In some embodiments, the processing device may further be configured to generate a reminder in response to a determination that the difference exceeds the threshold.

In some embodiments, the storage device may be integrated into the imaging device.

In some embodiments, the storage device may be separate from the imaging device.

In some embodiments, the processing device may further be configured to generate a first image of the portion of the subject based on the image data generated by the imaging device. The processing device may further be configured to determine a second image by performing an augmented reality processing operation on the first image. The interaction device may be configured to display the second image on a body surface of the subject corresponding to the portion of the subject.

In some embodiments, the processing device may further be configured to cause a medical operation to be performed on the portion of the subject.

In some embodiments, the processing device may be operably connected to an arm.

In some embodiments, the processing device may be operably connected to at least one operating element of the arm. The at least one operating element may include at least one of a scalpel or a puncture needle.

In some embodiments, the imaging device may be an X-ray imaging device, a computed tomography (CT) device, a magnetic resonance imaging (MRI) device, a positron emission tomography (PET) device, an ultrasound device, or a multi-modality device.

In some embodiments, the X-ray imaging device may be a mobile digital radiography (DR) or a C-arm device.

In some embodiments, the CT device may be a cone beam breast computed tomography (CBCT).

According to an aspect of the present disclosure, a system is provided. The system may include an imaging device, a processing device, and an interaction device. The imaging device may be configured to generate image data by imaging a subject or a portion thereof. The processing device may be configured to communicate with the imaging device and the interaction device. The processing device may be configured to generate a first image of the portion of the subject based on the image data generated by the imaging device. The processing device may be configured to determine a second image by performing an augmented reality processing operation on the first image. The interaction device may be configured to display the second image on a body surface of the subject corresponding to the portion of the subject.

In some embodiments, the processing device may further be configured to cause a medical operation to be performed on the portion of the subject.

In some embodiments, the processing device may be operably connected to an arm.

In some embodiments, the processing device may be operably connected to at least one operating element of the arm. The at least one operating element of the arm may include at least one of a scalpel or a puncture needle.

In some embodiments, the interaction device may include at least one of an optical device or a projection device.

In some embodiments, the system may include a storage device configured to store information regarding a plurality of preset postures. The interaction device, in communication with the storage device, may be configured to display information regarding at least one preset posture of the plurality of preset postures.

In some embodiments, the interaction device may include a holographic projector configured to project a first image of the at least one preset posture of the plurality of preset postures.

In some embodiments, the holographic projector may be movable.

In some embodiments, the system may include a movable base configured to carry the holographic projector to move.

In some embodiments, the system may include a control device, in communication with the interaction device, configured to control the interaction device.

In some embodiments, the processing device may further be configured to determine a display position of the information regarding the at least one preset posture based on a position of the imaging device.

In some embodiments, the system may include an image capture device configured to capture a second image representing an actual posture of the subject when the subject is positioned within the imaging device.

In some embodiments, the processing device may further be configured to determine a difference between the actual posture of the subject and the at least one preset posture of the subject. The processing device may further be configured to determine whether the difference is below a threshold.

In some embodiments, the processing device may further be configured to generate a reminder in response to a determination that the difference exceeds the threshold.

In some embodiments, the storage device may be integrated into the imaging device.

In some embodiments, the storage device may be separate from the imaging device.

In some embodiments, the imaging device may be an X-ray imaging device, a CT device, an MR device, a PET device, an ultrasound device, or a multi-modality device.

In some embodiments, the X-ray imaging device may be a mobile digital radiography (DR) or a C-arm device.

In some embodiments, the CT device may be a cone beam breast computed tomography (CBCT).

According to still another aspect of the present disclosure, a method may be implemented on a computing device having one or more processors and one or more storage devices. The method may include generating a first image of at least a portion of a subject based on image data generated by an imaging device. The method may include determining a second image by performing an augmented reality processing operation on the first image. The method may include displaying the second image on a body surface of the subject corresponding to the at least the portion of the subject.

In some embodiments, the first image may include information associated with a plurality of types of tissue of the subject. The method may include displaying the plurality of types of tissue of the subject in the first image distinguishably.

In some embodiments, the method may include displaying the plurality of types of tissue of the subject in the first image in different colors, different grayscales, or different textures.

In some embodiments, the first image may be a three-dimensional image. The method may include obtaining coordinates of each type of tissue of the plurality of types of tissue in the first image in a three-dimensional coordinate system. The method may include adjusting the first image based on the coordinates of the each type of tissue in the first image. The method may include determining the second image by aligning the adjusted first image and the body surface of the subject corresponding to the portion of the subject.

In some embodiments, the method may include displaying indication information on the body surface of the subject.

In some embodiments, the indication information may include at least one of a preset slit position, a preset needle insertion direction, a preset puncture path, or a preset puncture needle position.

According to still another aspect of the present disclosure, a system may include a computer-readable storage medium storing executable instructions, and at least one processor in communication with the computer-readable storage medium. When executing the executable instructions, the at least one processor may cause the system to implement a method. The method may include generating a first image of at least a portion of a subject based on image data generated by an imaging device. The method may include determining a second image by performing an augmented reality processing operation on the first image. The method may include displaying the second image on a body surface of the subject corresponding to the at least the portion of the subject.

According to still another aspect of the present disclosure, a non-transitory computer readable medium may store instructions. The instructions, when executed by at least one processor, may cause the at least one processor to implement a method. The method may include generating a first image of at least a portion of a subject based on image data generated by an imaging device. The method may include determining a second image by performing an augmented reality processing operation on the first image. The method may include displaying the second image on a body surface of the subject corresponding to the at least the portion of the subject.

According to an aspect of the present disclosure, a system is provided. The system may include a medical device, a storage device, a processing device, and an interaction device. The medical device may be configured to perform a medical procedure on a subject or a portion thereof. The storage device may be configured to store subject procedure information relating to the medical procedure on the subject. The processing device may be configured to communicate with the medical device, the storage device, and the interaction device. The interaction device, in communication with the storage device, may be configured to communicate to the subject at least a portion of the subject procedure information during the medical procedure.

In some embodiments, the interaction device may include at least one of an optical device, a projection device, and an audio device.

In some embodiments, the interaction device may include a holographic projector configured to project the at least the portion of the subject procedure information.

In some embodiments, the holographic projector may be movable.

In some embodiments, the system may include a movable base configured to carry the holographic projector to move.

In some embodiments, the system may include a control device, in communication with the interaction device, configured to control the interaction device.

In some embodiments, the processing device may further be configured to determine a display position of the at least the portion of the subject procedure information based on a position of the imaging device.

In some embodiments, the subject procedure information relating to the medical procedure may include at least one of information regarding a plurality of preset positions, information regarding a plurality of preset postures, and breath information.

In some embodiments, the storage device may be integrated into the imaging device.

In some embodiments, the storage device may be separate from the imaging device.

In some embodiments, the processing device may further be configured to generate a first image of the portion of the subject based on the image data generated by the imaging device. The processing device may further be configured to determine a second image by performing an augmented reality processing operation on the first image. The interaction device may be configured to display the second image on a body surface of the subject corresponding to the portion of the subject.

In some embodiments, the processing device may further be configured to cause a medical operation to be performed on the the portion of the subject.

In some embodiments, the processing device may be operably connected to an arm.

In some embodiments, the processing device may be operably connected to at least one operating element of the arm. The at least one operating element may include at least one of a scalpel or a puncture needle.

In some embodiments, the imaging device may be an X-ray imaging device, a computed tomography (CT) device, a magnetic resonance imaging (MRI) device, a positron emission tomography (PET) device, an ultrasound device, or a multi-modality device.

In some embodiments, the X-ray imaging device may be a mobile digital radiography (DR) or a C-arm device.

In some embodiments, the CT device may be a cone beam breast computed tomography (CBCT).

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of an exemplary computing device on which the processing device may be implemented according to some embodiments of the present disclosure;

FIG. 3 is a schematic diagram illustrating exemplary hardware and/or software components of an exemplary mobile device on which the terminal(s) may be implemented according to some embodiments of the present disclosure;

FIG. 4 is a schematic diagram illustrating an exemplary interaction device according to some embodiments of the present disclosure;

FIG. 5 is a schematic diagram illustrating an exemplary processing device according to some embodiments of the present disclosure;

FIG. 6 is a flowchart illustrating an exemplary process for positioning a subject according to some embodiments of the present disclosure;

FIG. 7 is a schematic diagram illustrating an exemplary processing device according to some embodiments of the present disclosure;

FIG. 8 is a schematic diagram illustrating an exemplary processing device according to some embodiments of the present disclosure;

FIG. 9 is a flowchart illustrating an exemplary process for displaying a second image on a body surface of a subject according to some embodiments of the present disclosure;

FIG. 10 is a flowchart illustrating an exemplary process for determining a second image according to some embodiments of the present disclosure; and

FIG. 11 is a flowchart illustrating an exemplary process for displaying a second image on a body surface of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Also, the term “exemplary” is intended to refer to an example or illustration.

It will be understood that the terms “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

Spatial and functional relationships between elements are described using various terms, including “connected,” “attached,” and “mounted.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, attached, or positioned to another element, there are no intervening elements present. Other words used to describe the relationship between elements should be interpreted in a like fashion (e.g., “between,” versus “directly between,” “adjacent,” versus “directly adjacent,” etc.).

It should also be understood that terms such as “top,” “bottom,” “upper,” “lower,” “vertical,” “lateral,” “above,” “below,” “upward(s),” “downward(s),” “left-hand side,” “right-hand side,” “horizontal,” and other such spatial reference terms are used in a relative sense to describe the positions or orientations of certain surfaces/parts/components of the imaging system with respect to other such features of the imaging system when the imaging device is in a normal operating position and may change if the position or orientation of the imaging system changes.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

For illustration purposes, the following description is provided to help better understanding a positioning process. It is understood that this is not intended to limit the scope of the present disclosure. For persons having ordinary skills in the art, a certain amount of variations, changes and/or modifications may be deducted under the guidance of the present disclosure. Those variations, changes and/or modifications do not depart from the scope of the present disclosure.

An aspect of the present disclosure relates to a system. The system may include a medical device (e.g., an imaging device, a treatment device), a storage device, a processing device, and an interaction device (e.g., an optical device, a projection device, an audio device). The storage device may store information regarding a plurality of preset postures suitable for imaging of a subject (e.g., a patient) using the medical device. At least one preset posture may be selected from the plurality of preset postures based on information associated with the subject. Information regarding the selected at least one preset posture may be transmitted to the interaction device. The interaction device may provide to the subject the information regarding the selected at least one preset posture of the plurality of preset postures. Accordingly, the subject may be positioned based on the information regarding the at least one preset posture of the plurality of preset postures. For example, the interaction device may project an image of the selected at least one preset posture in a space (e.g., a scanning room). The subject may adjust his or her posture according to the projected image of the selected at least one preset posture. After the subject is positioned, the medical device may perform a medical procedure on the subject, e.g., generate image data by imaging the subject or a portion thereof. Therefore, the user's instruction time may be reduced, the positioning process may be simplified, and accordingly the efficiency of the positioning process may be improved. A deviation of an actual posture of the subject, compared to the at least one preset posture, caused by the user personally guiding the subject may be avoided, and accordingly the accuracy of the imaging and/or treatment process may be improved, and the imaging quality may also be ensured.

In another aspect of the present disclosure, the processing device may generate a first image of at least a portion of the subject based on the image data generated by the imaging device. The processing device may also determine a second image by performing an augmented reality processing operation on the first image. The interaction device may display the second image on a body surface of the subject corresponding to the at least the portion of the subject. Therefore, the user may simultaneously view the second image (e.g., a three-dimensional image) associated with the portion of the subject, and an interior structure of the subject, or a portion thereof, displayed on the body surface of the subject, and a surrounding environment (e.g., a medical instrument) of the portion of the subject. With the assistance of AR visualization, the operation of the medical instrument may be simplified, automated and/or semi-automated, and accordingly the efficiency and/or the accuracy of the operation may be improved, which may reduce trauma to the subject from the operation and/or avoid excessive imaging accompanying the operation.

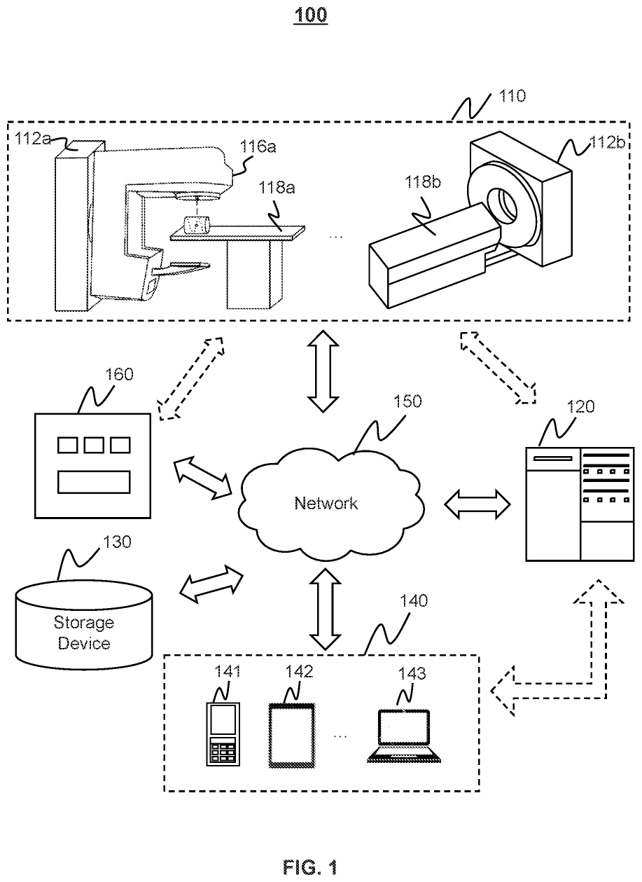

FIG. 1 is a schematic diagram illustrating an exemplary imaging system according to some embodiments of the present disclosure. As shown, the imaging system 100 may include a medical device 110, a processing device 120, a storage device 130, one or more terminal(s) 140, a network 150, and an interaction device 160. In some embodiments, the medical device 110, the processing device 120, the storage device 130, the terminal(s) 140, and/or the interaction device 160 may be connected to and/or communicate with each other via a wireless connection (e.g., the network 150), a wired connection, or a combination thereof. The imaging system 100 may include various types of connection between its components. For example, the medical device 110 may be connected to the processing device 120 through the network 150, or connected to the processing device 120 directly as illustrated by the bidirectional dotted arrow connecting the medical device 110 and the processing device 120 in FIG. 1. As another example, the storage device 130 may be connected to the processing device 120 through the network 150, as illustrated in FIG. 1, or connected to the processing device 120 directly. As still another example, the terminal(s) 140 may be connected to the processing device 120 through the network 150, or connected to the processing device 120 directly as illustrated by the bidirectional dotted arrow connecting the terminal(s) 140 and the processing device 120 in FIG. 1. As still another example, the terminal(s) 140 may be connected to the medical device 110 through the network 150, as illustrated in FIG. 1, or connected to the medical device 110 directly. As still another example, the interaction device 160 may be connected to the medical device 110 through the network 150, or connected to the medical device 110 directly as illustrated by the bidirectional dotted arrow connecting the medical device 110 and the interaction device 160 in FIG. 1.

The medical device 110 may be configured to perform a medical procedure (e.g., an imaging operation, a treatment operation) on a subject or a portion thereof. In some embodiments, the medical device 110 may be a radiation therapy (RT) device. In some embodiments, the RT device may deliver a radiation beam to a subject (e.g., a patient) or a portion thereof. In some embodiments, the RT device may include a linear accelerator (also referred to as “linac”). The linac may generate and emit a radiation beam (e.g., an X-ray beam) from a treatment head 116a. The radiation beam may pass through one or more collimators (e.g., a multi-leaf collimator (MLC)) of certain shapes, and enter into the subject. In some embodiments, the radiation beam may include electrons, photons, or other types of radiation. In some embodiments, the energy of the radiation beam may be in the megavoltage range (e.g., >1 MeV), and may therefore be referred to as a megavoltage beam. The treatment head 116a may be coupled to a gantry 112a. The gantry 112a may rotate, for example, clockwise or counter-clockwise around a gantry rotation axis. In some embodiments, the treatment head 116a may rotate along with the gantry 112a. In some embodiments, the RT device may include a table 118a configured to support the subject during radiation treatment.

In some embodiments, the medical device 110 may be an imaging device. The imaging device may generate or provide image(s) via imaging a subject or a part of the subject. In some embodiments, the imaging device may be a medical imaging device, for example, a computed tomography (CT) device, a magnetic resonance imaging (MRI) device, a positron emission tomography (PET) device, an ultrasound device, an X-ray imaging device, a digital subtraction angiography (DSA) device, a dynamic spatial reconstruction (DSR) device, a multimodality device, or the like, or any combination thereof. Exemplary X-ray imaging devices may include a suspended X-ray imaging device, a digital radiography (DR) device (e.g., a mobile digital X-ray imaging device), a C-arm device, or the like. Exemplary CT devices may include a cone beam breast computed tomography (CBCT), or the like. In some embodiments, the imaging device may include a gantry 112b to support one or more imaging components configured to imaging the subject, and/or a table 118b configured to support the subject during an imaging process. In some embodiments, the imaging device may include a single-modality scanner. The single-modality scanner may include an MRI scanner, a CT scanner, a PET scanner, or the like, or any combination thereof. In some embodiments, the imaging device may include a multi-modality scanner. The multi-modality scanner may include a positron emission tomography-computed tomography (PET-CT) scanner, a positron emission tomography-magnetic resonance imaging (PET-MRI) scanner, or the like, or any combination thereof. In some embodiments, the imaging device may transmit the image(s) via the network 150 to the processing device 120, the storage device 130, the interaction device 160, and/or the terminal(s) 140. For example, the image(s) may be sent to the processing device 120 for further processing or may be stored in the storage device 130.

In some embodiments, the medical device 110 may be an integrated device of an imaging device and an RT device. In some embodiments, the medical device 110 may include one or more surgical instruments. In some embodiments, the medical device 110 may include an operating table (or table for brevity) configured to support a subject during surgery. The table 118a or 118b may support a subject during a treatment process or imaging process, and/or support a phantom during a correction process of the medical device 110. The table 118a or 118b may be adjustable and/or movable to suit for different application scenarios.

In some embodiments, the subject to be treated or imaged may include a body, substance, or the like, or any combination thereof. In some embodiments, the subject may include a specific portion of a body, such as a head, a thorax, an abdomen, or the like, or any combination thereof. In some embodiments, the subject may include a specific organ, such as a breast, an esophagus, a trachea, a bronchus, a stomach, a gallbladder, a small intestine, a colon, a bladder, a ureter, a uterus, a fallopian tube, etc. In the present disclosure, “object” and “subject” are used interchangeably.

The processing device 120 may process data and/or information obtained from the medical device 110, the storage device 130, the terminal(s) 140, and/or the interaction device 160. For example, the processing device 120 may generate a first image of a portion of a subject by image reconstruction using image data generated by the medical device 110. As another example, the processing device 120 may determine a second image by performing an augmented reality processing operation on a first image of a portion of a subject. As a further example, the processing device 120 may cause a medical operation to be performed on a portion of a subject. As a further example, the processing device 120 may determine a display position of an image of at least one preset posture based on a position of the medical device 110. As a still further example, the processing device 120 may determine whether a difference between an actual posture of a subject and a preset posture of the subject is below a threshold. As a still further example, the processing device 120 may generate a reminder in response to a determination that a difference between an actual posture of a subject and a preset posture of the subject exceeds a threshold. In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, the terminal(s) 140, and/or the interaction device 160 via the network 150. As another example, the processing device 120 may be directly connected to the medical device 110, the terminal(s) 140, the storage device 130, and/or the interaction device 160 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal 140. In some embodiments, the processing device 120 may be part of the medical device 110. In some embodiments, the processing device 120 may be part of the interaction device 160.

The storage device 130 may store data, instructions, and/or any other information. In some embodiments, the storage device 130 may store data obtained from the medical device 110, the processing device 120, the terminal(s) 140, and/or the interaction device 160. For example, the storage device 130 may store information associated with a subject. The information associated with the subject may include a name of the subject, the gender of the subject, the age of the subject, a size of the subject (e.g., a height of the subject, a weight of the subject), a portion of the subject to be imaged, or the like, or any combination thereof. As another example, the storage device 130 may store subject procedure information relating to a medical procedure (e.g., an imaging operation, a treatment operation) on a subject. The subject procedure information may include information regarding a plurality of preset positions, information regarding a plurality of preset postures, breath information, or the like, or any combination thereof. As used herein, the breath information of the subject may refer to information regarding how the subject should breath during the medical procedure. The breath information may include a breathing count, a breathing rate, or the like, or any combination thereof. As still another example, the storage device 130 may store an image representing a status of a subject obtained from an image capture device. The status of the subject may include a position of the subject, a posture of the subject, or the like, or any combination thereof. As a further example, the storage device 130 may store a first image of a portion of a subject generated by reconstructing image data collected by the medical device 110. As a further example, the storage device 130 may store a second image generated by performing an augmented reality processing operation on a first image of a portion of a subject. In some embodiments, the storage device 130 may store data and/or instructions that the processing device 120 and/or the terminal 140 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform as described elsewhere in the disclosure.

In some embodiments, the storage device 130 may be connected to the network 150 to communicate with one or more other components in the imaging system 100 (e.g., the processing device 120, the terminal(s) 140, and/or the interaction device 160). One or more components in the imaging system 100 may access the data or instructions stored in the storage device 130 via the network 150. In some embodiments, the storage device 130 may be integrated into the medical device 110.

The terminal(s) 140 (also referred to as a control device) may be connected to and/or communicate with the medical device 110, the processing device 120, the storage device 130, and/or the interaction device 160. For example, a user may provide an input via a user interface implemented on the terminal 140. The input may include an imaging parameter (e.g., a current of an imaging device, a voltage of an imaging device, a scan time), an image construction parameter, information associated with a subject to be imaged or treated, subject procedure information, as described elsewhere in the present disclosure. As another example, the terminal 140 may receive an instruction provided by the user for controlling the imaging or the treatment of the subject by the medical device 110. As still another example, the terminal 140 may control the storage device 130 to transmit information regarding at least one preset posture to the interaction device 160 for display. As a further example, the terminal 140 may control the interaction device 160 to obtain information regarding at least one preset posture from the storage device 130 for display.

In some embodiments, the terminal 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the imaging system 100. In some embodiments, one or more components of the imaging system 100 (e.g., the medical device 110, the processing device 120, the storage device 130, the terminal(s) 140, the interaction device 160, etc.) may communicate information and/or data with one or more other components of the imaging system 100 via the network 150. For example, the processing device 120 and/or the terminal 140 may obtain image data from the medical device 110 via the network 150. As another example, the processing device 120 and/or the terminal 140 may obtain information stored in the storage device 130 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a W-Fi network, etc.), a cellular network (e.g., a Long Term Evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the imaging system 100 may be connected to the network 150 to exchange data and/or information.

The interaction device 160 may be configured to display data and/or information associated with the imaging system 100. In some embodiments, the interaction device 160 may communicate to a subject at least a portion of subject procedure information during a medical procedure. For example, the interaction device 160 may provide to a subject information regarding at least one preset posture of a plurality of preset postures. In some embodiments, the interaction device 160 may provide to a user information regarding a subject during a medical operation. For example, the interaction device 160 may display a second image on a body surface of the subject corresponding to a portion of the subject to guide an operation by a user.

In some embodiments, the interaction device 160 may include an optical device, a projection device, an audio device, or the like, or any combination thereof. The optical device may include a virtual reality device, an augmented reality device, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™ a RiftCon™, a Fragments™, a Gear VR™, etc.

The projection device may include a digital light processing (DLP) projector, a liquid crystal display (LCD) projector, or the like, or any combination thereof. In some embodiments, the projection device may include a holographic projector. The holographic projector may use holograms rather than graphic images to produce projected pictures. The holographic projector may shine special white light or laser light onto or through holograms. The projected light may produce a bright two-dimensional or three-dimensional image.

In some embodiments, the interaction device 160 may be installed close to the medical device 110. In some embodiments, the medical device 110 and the interaction device 160 may be installed in a specific imaging space. For example, the imaging space may be a scanning room.

According to some embodiments of the present disclosure, the imaging system 100 may generate image data by imaging a portion of a subject. The user may diagnose the subject based on at least a portion of the image data. Before and/or during the imaging process, the imaging system 100 of the present disclosure may display an image of at least one preset posture for the subject. The subject may imitate the at least one preset posture to reduce the guide time of the user, which may improve the efficiency and/or the accuracy of the positioning process. In addition, a deviation of an actual posture of the subject, compared to the at least one preset posture of the subject, caused by the user personally guiding the subject may be avoided, which may ensure the imaging quality of the imaging system 100.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the storage device 130 may be data storage including cloud computing platforms, such as a public cloud, a private cloud, community, and hybrid clouds, or the like.

In some embodiments, the imaging system 100 may further include an image capture device configured to acquire image data (e.g., a video, an image) of a subject to be imaged and/or treated. In some embodiments, the image capture device may be configured to capture an image representing a status of the subject when the subject is positioned within the medical device 110. The status of the subject may include a position of the subject, a posture of the subject, or the like. In some embodiments, the image capture device may be mounted on the medical device 110. In some embodiments, the image capture device may be mounted on the interaction device 160. In some embodiments, the image capture device may be and/or include any suitable device that is capable of acquiring image data. Exemplary image capture devices may include a camera (e.g., a digital camera, an analog camera, etc.), a scanner, a video recorder, a mobile phone, a tablet computing device, a wearable computing device, an infrared imaging device (e.g., a thermal imaging device), or the like.

In some embodiments, the imaging system 100 may further include a positioning device. The positioning device may be configured to determine a position of at least one component (e.g., a detector of an imaging device) of the medical device 110. For example, the positioning device, in communication with the processing device 120, may determine the position of the at least one component (e.g., a detector) of the medical device 110 in real time. The display position of the at least one preset posture may be determined based on the real-time position of the at least one component (e.g., a detector) of the medical device 110, which may avoid or reduce a deviation in the display position, compared to an intended display position, caused by the position change of the at least one component (e.g., a detector) of the medical device 110. In some embodiments, the positioning device may include a position sensor.

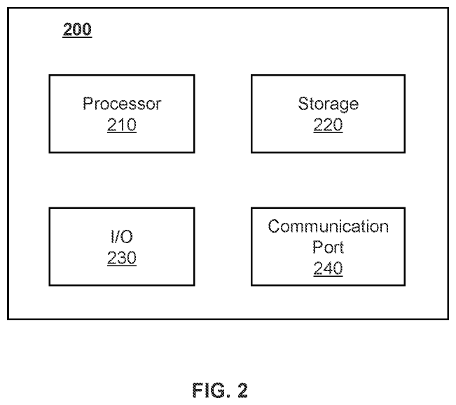

FIG. 2 is a schematic diagram illustrating exemplary hardware and/or software components of an exemplary computing device 200 on which the processing device 120 may be implemented according to some embodiments of the present disclosure. As illustrated in FIG. 2, the computing device 200 may include a processor 210, a storage 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process imaging data obtained from the medical device 110, the terminal(s) 140, the storage device 130, and/or any other component of the imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the medical device 110, the terminal(s) 140, the storage device 130, the interaction device 160, and/or any other component of the imaging system 100. The storage 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the terminal(s) 140, the interaction device 160, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

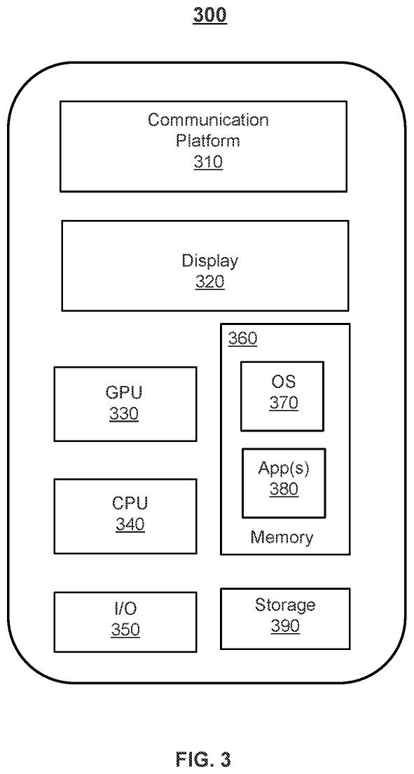

FIG. 3 is a schematic diagram illustrating exemplary hardware and/or software components of an exemplary mobile device 300 on which the terminal(s) 140 may be implemented according to some embodiments of the present disclosure.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the imaging system 100, and enable data and/or signal to be transmitted between the mobile device 300 and other components of the imaging system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the imaging system 100. For example, the communication platform 310 may transmit data and/or signals inputted by a user to other components of the imaging system 100. The inputted data and/or signals may include a user instruction. As another example, the communication platform 310 may receive data and/or signals transmitted from the processing device 120. The received data and/or signals may include imaging data acquired by a detector of the medical device 110.

In some embodiments, a mobile operating system (OS) 370 (e.g., iOS™, Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information respect to an imaging process or other information from the processing device 120. User interactions with the information stream may be achieved via the I/O 350 and provided to the processing device 120 and/or other components of the imaging system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

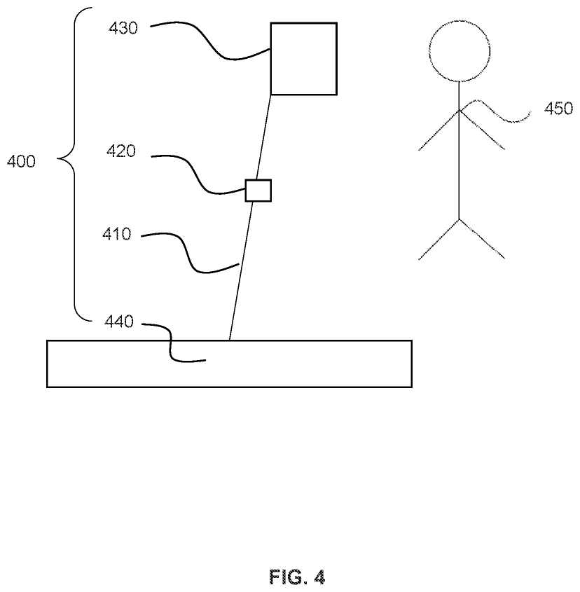

FIG. 4 is a schematic diagram illustrating an exemplary interaction device according to some embodiments of the present disclosure. In some embodiments, an interaction device 400 may be an example of the interaction device 160 or a portion of the interaction device 160. As shown in FIG. 4, the interaction device 400 may include a frame 410, a receiving device 420, a projection device 430, and a movable base 440.

The frame 410 may be configured to support one or more components (e.g., the receiving device 420, the projection device 430) of the interaction device 400. In some embodiments, the frame 410 may be mounted on the ground. In some embodiments, the frame 410 may be mounted on a wall.

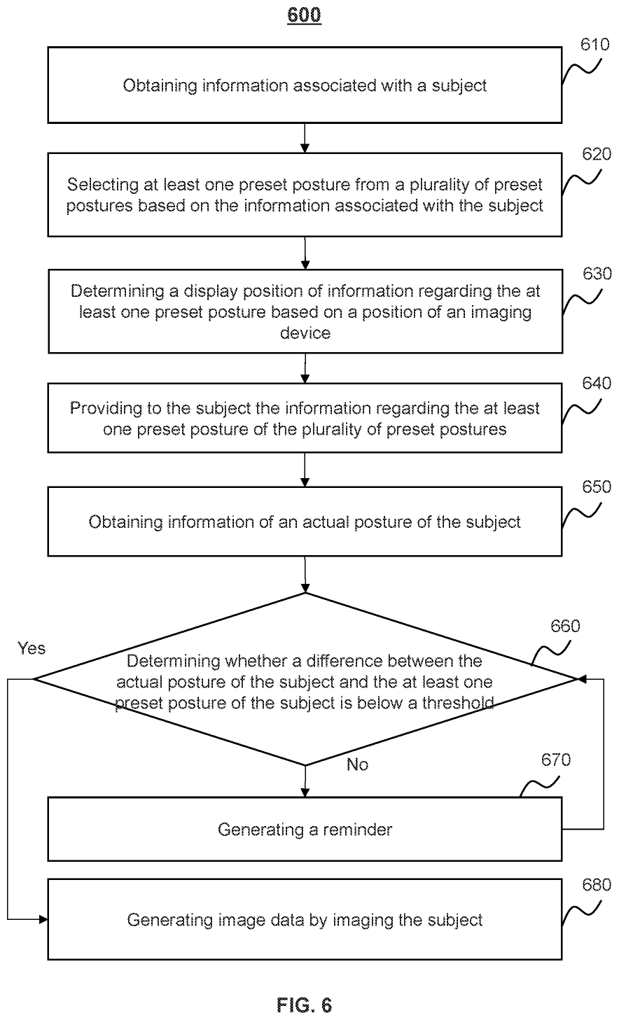

The receiving device 420 may be configured to obtain information regarding the procedure to be performed in the medical device 110, e.g., at least one preset posture of a subject to be imaged in the medical device 110. In some embodiments, information regarding a plurality of preset postures of a subject to be imaged in the medical device 110 may be stored in a storage device (e.g., the storage device 130) of the imaging system 100 or an external storage device. The receiving device 420 may access the storage device and retrieve information regarding the at least one preset posture based on information associated with the subject to be imaged and/or an applicable imaging protocol. More descriptions of the determination of the at least one preset posture may be found elsewhere in the present disclosure (e.g., FIG. 6, and descriptions thereof).

In some embodiments, the receiving device 420 may be mounted on the frame 410, as illustrated in FIG. 4. In some embodiments, the receiving device 420 may be separate from the frame 410.

The projection device 430 may be configured to display subject procedure information relating to a medical procedure on a subject, e.g., at least one preset posture (e.g., a posture 450) of a subject to be imaged in the medical device 110. For example, the projection device 430 may project an image of the at least one preset posture in a space for the subject to view. In some embodiments, the projection device 430 may be a holographic projector. The holographic projector may project a three-dimensional image (e.g., a three-dimensional posture) using lights in a space according to the holographic projection technology.

In some embodiments, the projection device 430 may be movable. For example, the projection device 430 may be movable mounted on the frame 410. Accordingly, a display position of the projected three-dimensional image of the preset posture may be adjusted by adjusting a position of the projection device 430, which may be convenient for the subject to view. For example, when the subject needs to lie on a table (e.g., the table 118a, the table 118b) for imaging or treatment, the projection device 430 may project the image of the at least one preset posture in a space above the subject. The subject may lie on the table to view the image of the at least one preset posture and imitate the at least one preset posture by adjusting his/her own current posture. As another example, when the subject needs to stand on the ground for imaging or treatment, the projection device 430 may project the image of the at least one preset posture in a space in front of the subject. The subject may stand on the ground to view the image of the at least one preset posture and imitate the at least one preset posture by adjusting his/her own current posture. In some embodiments, the projection device 430 may be movably suspend on the frame 410, which may save space.

The movable base 440 may be configured to carry the projection device 430 so that the projection device 430 may move. For example, when the projection device 430 is not in use, the projection device 430 may be moved to a storage location carried by the movable base 440. In some embodiments, the movable base 440 may be adapted to work collaboratively with a plurality of imaging devices and/or treatment devices. When used with an imaging device of the plurality of imaging devices to perform a scan, the projection device 430 may be moved to a location near the imaging device and operably connected to the imaging device to project the image of the at least one preset posture. After the scan is finished, the projection device 430 may be moved to a location close to another imaging device of the plurality of imaging devices to perform similar operations.

In some embodiments, the interaction device 400 may include a controller (not shown in FIG. 4). The controller may be configured to control the projection device 430. For example, the controller may include a switch configured to control a status of the projection device 430. The status of the projection device 430 may include an open status, a closed status, a standby status, or the like.

In some embodiments, the interaction device 400 may include an image capture device. The image capture device may be configured to acquire image data (e.g., a video, an image) of the subject to be imaged and/or treated. In some embodiments, the image capture device may be configured to capture an image representing an actual posture and/or an actual position of the subject. In some embodiments, the image capture device may be mounted on the frame 410. In some embodiments, the image capture device may be and/or include any suitable device that is capable of acquiring image data. Exemplary image capture devices may include a camera (e.g., a digital camera, an analog camera, etc.), a scanner, a video recorder, a mobile phone, a tablet computing device, a wearable computing device, an infrared imaging device (e.g., a thermal imaging device), or the like.

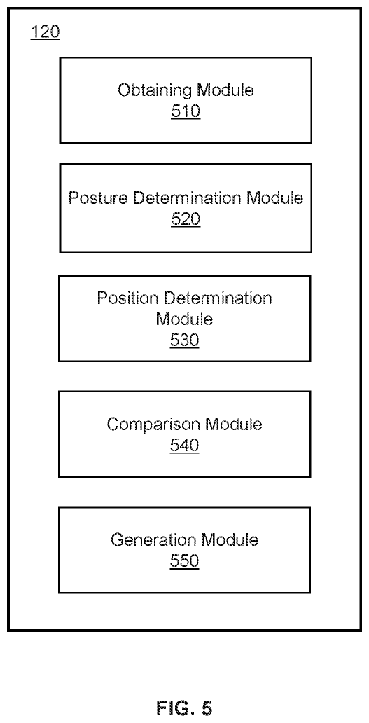

In some embodiments, the interaction device 400 may include a processor (not shown in FIG. 4). In some embodiments, the processor may include a position determination module (e.g., a position determination module 530), a comparison module (e.g., a comparison module 540), and a generation module (e.g., the generation module 550). The position determination module may be configured to determine a display position of information regarding the at least one preset posture based on a position of an imaging device (e.g., the medical device 110). The comparison module may be configured to determine whether a difference between the actual posture of the subject and the at least one preset posture of the subject is below a threshold. The generation module may be configured to generate a reminder in response to a determination that the difference between the actual posture of the subject and the at least one preset posture of the subject exceeds the threshold. More descriptions of the position determination module, the comparison module, and the generation module may be found elsewhere in the present disclosure (e.g., FIG. 5, and descriptions thereof).

In some embodiments, the receiving device 420, the projection device 430, the controller, the image capture device, the processor, and/or one or more other components of the imaging system 100 may be connected to and/or communicate with each other via a wireless connection (e.g., a Wi-Fi, a Bluetooth, a radio frequency transmission, an infrared transmission), a wired connection, or a combination thereof. For example, the receiving device 420 may be connected to the storage device 130 via the network 150 or a cable. The information regarding the at least one preset posture stored in the storage device 130 may be transmitted to the receiving device 420. As another example, the projection device 430 may be operably connected to the receiving device 420 via the network 150. The information regarding the at least one preset posture obtained by the receiving device 420 may be transmitted to the projection device 430 for display. As still another example, the projection device 430 may be operably connected to a control device (e.g., the terminal 140) via the network 150. The projection device 430 may operate based on an instruction provided by the user and transmitted to via the control device. As a further example, the projection device 430 may be operably connected to the control device via the receiving device 420.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more components of the interaction device 400 may be integrated into a single component. For example, the receiving device 420, the controller, and the processor may be integrated into a single component. In some embodiments, one or more components may be omitted in the interaction device 400. For example, the movable base 440 may be omitted in the interaction device 400. The movable base 440 may be integrated into the medical device 110. The interaction device 400 may be connected to the imaging system 100 via the movable base 440.

FIG. 5 is a schematic diagram illustrating an exemplary processing device according to some embodiments of the present disclosure. In some embodiments, the processing device 120 may include an obtaining module 510, a posture determination module 520, a position determination module 530, a comparison module 540, and a generation module 550. The modules may be hardware circuits of at least part of the processing device 120. The modules may also be implemented as an application or set of instructions read and executed by the processing device 120. Further, the modules may be any combination of the hardware circuits and the application/instructions. For example, the modules may be part of the processing device 120 when the processing device 120 is executing the application or set of instructions.

The obtaining module 510 may be configured to obtain data and/or information associated with the imaging system 100. For example, the obtaining module 510 may obtain information associated with a subject. As another example, the obtaining module 510 may obtain an image of an actual posture of a subject and/or an image of an actual position of the subject. As still another example, the obtaining module 510 may obtain image data associated with a portion of a subject. In some embodiments, the obtaining module 510 may obtain the data and/or the information associated with the imaging system 100 from one or more components (e.g., the medical device 110, the storage device 130, the terminal 140, the interaction device 160, an image capture device) of the imaging system 100 via the network 150.

The posture determination module 520 may be configured to select at least one preset posture from a plurality of preset postures. For example, the posture determination module 520 may select the at least one preset posture from the plurality of preset postures based on a portion of a subject to be imaged, an imaging protocol, or the like, or a combination thereof. In some embodiments, information regarding a plurality of preset postures of a subject to be imaged in the medical device 110 may be stored in a storage device (e.g., the storage device 130) of the imaging system 100 or an external storage device. The processing device 120 may access the storage device and retrieve information regarding the at least one preset posture of the plurality of preset postures from the storage device.

The position determination module 530 may be configured to determine a display position of information regarding at least one preset posture. For example, the position determination module 530 may determine the display position of an image of the at least one preset posture based on a position of at least one component (e.g., a detector) of an imaging device (e.g., the medical device 110), a position of a subject, or the like, or a combination thereof. In some embodiments, the position determination module 530 may be a computer or a module with computing functions.

The comparison module 540 may be configured to compare data and/or information associated with the imaging system 100. In some embodiments, the comparison module 540 may compare an actual posture of a subject and a preset posture of the subject. For example, the comparison module 540 may determine a difference between an actual posture of a subject and a preset posture of the subject and compare the difference with a threshold. In some embodiments, the comparison module 540 may include a comparator.

The generation module 550 may be configured to generate data and/or information associated with the imaging system 100. For example, the generation module 550 may generate a reminder in response to a determination that a difference between an actual posture of a subject and a preset posture of the subject exceeds a threshold. As another example, the generation module 550 may generate a reminder in response to a determination that a difference between an actual posture of a subject and a preset posture of the subject is below a threshold. In some embodiments, the generation module 550 may generate a reminder in the form of text, voice, an image, a video, a haptic alert, or the like, or any combination thereof. In some embodiments, the generation module 550 may transfer a reminder to a terminal (e.g., the terminal 140) associated with the user. The terminal may convey the reminder to the user.

In some embodiments, one or more modules illustrated in FIG. 5 may be implemented in at least part of the imaging system 100 as illustrated in FIG. 1. For example, the obtaining module 510, the posture determination module 520, the position determination module 530, the comparison module 540, and the generation module 550 may be integrated into a console (not shown). Via the console, a user may set parameters for implementing operations described elsewhere in the present disclosure, e.g., an imaging procedure. In some embodiments, the modules illustrated in FIG. 5 may be implemented via the processing device 120 and/or the terminal 140.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the posture determination module 520 and the position determination module 530 may be combined into a single module, which may both determine at least one preset posture and a display position of information regarding the at least one preset posture. In some embodiments, one or more modules may be added or omitted in the processing device 120. For example, the processing device 120 may further include a storage module (not shown in FIG. 5) configured to store data and/or information (e.g., a plurality of preset postures) associated with the imaging system 100.

FIG. 6 is a flowchart illustrating an exemplary process for positioning a subject according to some embodiments of the present disclosure. In some embodiments, the process 600 may be implemented in the imaging system 100 illustrated in FIG. 1. For example, the process 600 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 610, the processing device 120 (e.g., the obtaining module 510) may obtain information associated with a subject. In some embodiments, the processing device 120 may obtain the information associated with the subject from a storage device (e.g., the storage device 130) of the imaging system 100 or an external storage device via the network 150.

In some embodiments, the subject may be a patient. In some embodiments, the information associated with the subject may include a name of the subject, the gender of the subject, the age of the subject, a size of the subject (e.g., a weight of the subject, a height of the subject), a portion of the subject to be imaged, or the like, or any combination thereof.

In 620, the processing device 120 (e.g., the posture determination module 520) may select at least one preset posture from a plurality of preset postures based on the information associated with the subject.

In some embodiments, the processing device 120 may select the at least one preset posture from the plurality of preset postures based on the portion of the subject to be imaged. For example, if the chest of the subject needs to be imaged, the subject may stand on the ground and put his/her hands on the waist. As another example, if the vertebral of the subject needs to be imaged, the subject may lie on a table with legs and arms slightly splayed on the table.

Additionally or alternatively, the processing device 120 may select the at least one preset posture from the plurality of preset postures based on an imaging protocol. In some embodiments, an imaging protocol may correspond to one or more preset postures of the plurality of preset postures. As used herein, an imaging protocol may refer to a combination of various imaging parameters (e.g., a collimator aperture, a detector aperture, an X-ray tube voltage and/or current, a scan mode, a table index speed, a gantry speed, a reconstruction field of view (FOV)), specific values or respective ranges of values of one or more imaging parameters, etc. In some embodiments, the imaging protocol may be determined based on hardware and software of a medical device (e.g., the medical device 110), a user's preference, and the information associated with the subject. When the medical device is used to perform a scan, the imaging protocol may be selected manually by the user or be determined by one or more components of the imaging system 100 according to different situations. The processing device 120 may determine the at least one preset posture corresponding to the selected imaging protocol from the plurality of preset postures.

In some embodiments, information regarding the plurality of preset postures may be stored in a storage device (e.g., the storage device 130) of the imaging system 100 or an external storage device. The plurality of preset postures may facilitate the acquisition of imaging data of satisfactory imaging quality by the medical device (e.g., the medical device 110). The processing device 120 may access the storage device and retrieve information regarding the at least one preset posture.

In 630, the processing device 120 (e.g., the position determination module 530) may determine a display position of information regarding the at least one preset posture based on a position of an imaging device.

In some embodiments, the processing device 120 may determine the display position of the information regarding the at least one preset posture (e.g., an image of the at least one preset posture) based on a position of at least one component (e.g., a detector) of the medical device (e.g., the medical device 110). For example, the portion of the subject to be imaged may be aligned to the position of the detector such that only the portion of the subject is imaged.

Additionally or alternatively, the processing device 120 may determine the display position of the image of the at least one preset posture based on a position of the subject. For example, when the subject lies on a table, the display position of the image of the at least one preset posture may be above the subject. As another example, when the subject stands on the ground, the display position of the image of the at least one preset posture may be in front of the subject. Accordingly, the display position of the image of the at least one preset posture may be convenient for the subject to view.

In some embodiments, the processing device 120 may determine the display position of the image of the at least one preset posture randomly. The processing device 120 may then adjust the position of the at least one component of the imaging device and/or the display position of the image of the at least one preset posture. In some embodiments, the processing device 120 may adjust the display position of the image of the at least one preset posture by adjusting the position of an interaction device (e.g., the interaction device 160) via a movable base (e.g., the movable base 440) of the interaction device.

In 640, the interaction device 160 may provide to the subject information regarding the at least one preset posture of the plurality of preset postures.

The interaction device 160 may include an optical device (e.g., a virtual reality device, an augmented reality device), a projection device (e.g., the projection device 430), an audio device, or the like, or any combination thereof, as described elsewhere in the present disclosure.

In some embodiments, the interaction device 160 (e.g., AR glasses) may display an image, or text regarding the at least one preset posture. In some embodiments, the interaction device 160 (e.g., the audio device) may play a recording regarding the at least one preset posture to direct the subject to imitate the at least one preset posture. In some embodiments, the interaction device 160 (e.g., the projection device 430) may project a three-dimensional image (e.g., a three-dimensional model of a human body) regarding the at least one preset posture in a space for the subject to view. For example, when the subject lies on a table for imaging, the interaction device 160 may project an image of the at least one preset posture in a space above the subject. As another example, when the subject stands on the ground for imaging, the interaction device 160 may project an image of the at least one preset posture in a space in front of the subject.

In 650, the processing device 120 (e.g., the obtaining module 510) may obtain information of an actual posture of the subject. In some embodiments, the processing device 120 may obtain an image representing the actual posture of the subject from an image capture device via the network 150. The image capture device may be and/or include any suitable device that is capable of acquiring an image as described elsewhere in the present disclosure (e.g., FIGS. 1, 4, and descriptions thereof).

In 660, the processing device 120 (e.g., the comparison module 540) may determine whether a difference between the actual posture of the subject and the at least one preset posture of the subject is below a threshold. In some embodiments, the processing device 120 may determine a degree of similarity between the actual posture of the subject and the preset posture of the subject. A higher degree of similarity between the actual posture of the subject and the preset posture of the subject may correspond to a smaller difference between the actual posture of the subject and the preset posture of the subject.

In some embodiments, the threshold may be a preset value or a preset range. The threshold may be a default parameter stored in a storage device (e.g., the storage device 130). Additionally or alternatively, the threshold may be set manually or determined by one or more components of the imaging system 100 according to different situations.

In response to a determination that the difference between the actual posture of the subject and the at least one preset posture of the subject exceeds the threshold, process 600 may proceed to operation 670. In 670, the processing device 120 (e.g., the generation module 550) may generate a reminder.

In some embodiments, the reminder may include information regarding an error in the actual posture of the subject. The error may indicate that which portion(s) of the actual posture does not match the at least one preset posture. In some embodiments, the processing device 120 may generate the reminder in the form of text, voice, a picture, a video, a haptic alert, or the like, or any combination thereof. In some embodiments, the processing device 120 may transfer the reminder to a terminal (e.g., the terminal 140) associated with the user. The terminal may convey the reminder to the user. For example, the terminal may display an image of a portion of the actual posture of the subject that does not match the at least one preset posture. As another example, the terminal may display text describing the portion of the actual posture of the subject that does not match the at least one preset posture.

In some embodiments, the operations 660 and 670 may be repeated until the difference between the actual posture of the subject and the at least one preset posture of the subject is below the threshold. In response to a determination that the difference between the actual posture of the subject and the at least one preset posture of the subject is below the threshold, process 600 may proceed to operation 680.

In 680, the medical device 110 may generate image data by imaging the subject.

In some embodiments, the processing device 120 may generate instructions to operate the medical device 110 to image the subject. For example, the processing device 120 may send instructions that cause the medical device 110 to adjust positions of one or more components of the medical device 110 to image the subject.

For illustration purposes, the medical device 110 may be an X-ray imaging device. A high voltage generator may generate a high-voltage and current for a tube. The tube may generate radiation beams (e.g., X-rays beams) of high-energetic photons. A detector may detect at least a portion of the radiation beams and generate data associated with the projection formed by the detected radiation beams (e.g., X-rays beams) as the image data.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, in response to a determination that the difference between the actual posture of the subject and the at least one preset posture of the subject is below the threshold, the processing device 120 may generate a reminder to inform the user that a scan can be performed. In some embodiments, two or more operations may be combined into a single operation. For example, operation 630 and operation 640 may be combined into a single operation.

In some embodiments, the processing device 120 may determine subject procedure information relating to a medical procedure on the subject based on the information associated with the subject, an imaging protocol, or the like, or a combination thereof. The subject procedure information may include information regarding at least one preset position of a plurality of preset positions, information regarding at least one preset posture of a plurality of preset postures, breath information, or the like, or any combination thereof, as described elsewhere in the present disclosure. The processing device 120 may communicate to the subject at least a portion of the subject procedure information during the medical procedure. For example, the processing device 120 may display an image or text regarding the at least one preset position, the at least one preset posture, and/or the breath information. As another example, the interaction device 160 (e.g., the audio device) may play a recording regarding the breath information to direct the subject to inhale and/or exhale during the medical procedure.

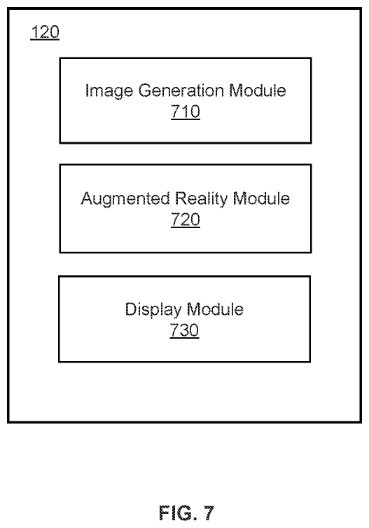

FIG. 7 is a schematic diagram illustrating an exemplary processing device according to some embodiments of the present disclosure. In some embodiments, the processing device 120 may include an image generation module 710, an augmented reality module 720, and a display module 730. The modules may be hardware circuits of at least part of the processing device 120. The modules may also be implemented as an application or set of instructions read and executed by the processing device 120. Further, the modules may be any combination of the hardware circuits and the application/instructions. For example, the modules may be part of the processing device 120 when the processing device 120 is executing the application or set of instructions.

The image generation module 710 may be configured to generate an image. For example, the image generation module 710 may generate a first image of a portion of a subject based on image data generated by a medical device (e.g., the medical device 110). In some embodiments, the image generation module 710 may reconstruct the image data to generate the first image based on one or more reconstruction techniques as described elsewhere in the present disclosure.

In some embodiments, the image generation module 710 may include computer hardware and computer programs that may reconstruct the image data to generate an image (e.g., a three-dimensional image). The computer hardware may include a storage device, a processor having image processing capabilities, or the like, or a combination thereof. The computer program may include three-dimensional image reconstruction software.

The augmented reality module 720 may be configured to perform an augmented reality processing operation on an image. In some embodiments, the augmented reality module 720 may determine a second image by performing an augmented reality processing operation on a first image of a portion of a subject. For example, the augmented reality module 720 may obtain coordinates of one or more types of tissue of a plurality of types of tissue, one or more organs, one or more feature points represented in a first image in a three-dimensional coordinate system. The augmented reality module 720 may adjust the first image based on such coordinates. The augmented reality module 720 may determine a second image by aligning the adjusted first image and a body surface of a subject corresponding to a portion of the subject.

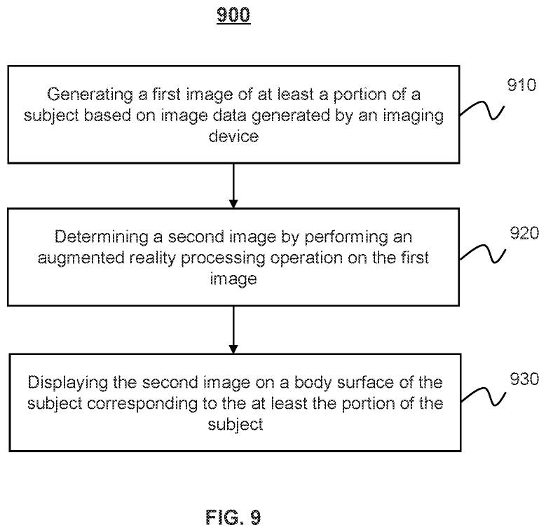

The display module 730 may be configured to display data and/or information associated with or generated by the imaging system 100. Data and/or information associated with the imaging system 100 may include information associated with a subject (e.g., a name of the subject, the gender of the subject, the age of the subject, a size of the subject (e.g., a height of the subject, a weight of the subject), a portion of the subject to be imaged), an imaging parameter associated with at least one component of the imaging system 100 (e.g., a current of a medical device, a voltage of a medical device, a scan time), or the like, or any combination thereof. Data and/or information generated by the imaging system 100 may include a first image of at least a portion of a subject, a second image generated by performing an AR operation on the first image, indication information, or the like, of any combination thereof. For example, the display module 730 may display a second image on a body surface of a subject corresponding to a portion of the subject. As another example, the display module 730 may display a plurality of types of tissue of a subject in a first image distinguishably (e.g., in different colors). As still another example, the display module 730 may display indication information on a body surface of a subject. More descriptions of the display module 730 may be found elsewhere in the present disclosure (e.g., FIG. 9, and descriptions thereof).

In some embodiments, one or more modules illustrated in FIG. 7 may be implemented in at least part of the imaging system 100 as illustrated in FIG. 1. For example, the image generation module 710, the augmented reality module 720, and the display module 730 may be integrated into a console (not shown). Via the console, a user may set parameters for implementing operations described elsewhere in the present disclosure. In some embodiments, the modules described in FIG. 7 may be implemented via the processing device 120 and/or the terminal 140.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be added or omitted in the processing device 120. For example, the processing device 120 may further include a storage module (not shown in FIG. 7) configured to store data and/or information (e.g., a first image, a second image) associated with the imaging system 100. As another example, the processing device 120 may further include a processing module (e.g., a processing module 840) configured to cause a medical operation to be performed on a portion of a subject.

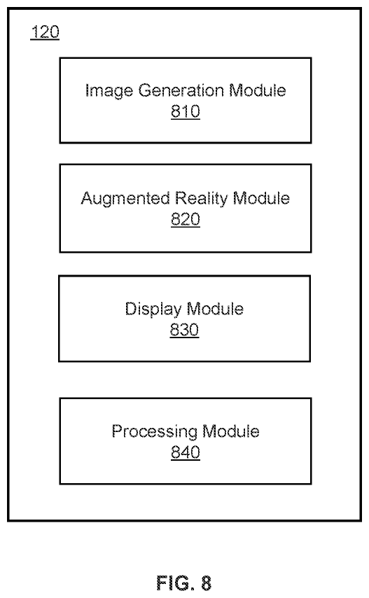

FIG. 8 is a schematic diagram illustrating an exemplary processing device according to some embodiments of the present disclosure. In some embodiments, the processing device 120 may include an image generation module 810, an augmented reality module 820, a display module 830, and a processing module 840. The modules may be hardware circuits of at least part of the processing device 120. The modules may also be implemented as an application or set of instructions read and executed by the processing device 120. Further, the modules may be any combination of the hardware circuits and the application/instructions. For example, the modules may be part of the processing device 120 when the processing device 120 is executing the application or set of instructions.

The image generation module 810 may be configured to generate an image. The image generation module 810 may be the same as or similar to the image generation module 710 as described elsewhere in the present disclosure (e.g., FIG. 7 and descriptions thereof).

The augmented reality module 820 may be configured to perform an augmented reality processing operation on an image. The augmented reality module 820 may be the same as or similar to the augmented reality module 720 as described elsewhere in the present disclosure (e.g., FIG. 7 and descriptions thereof).

The display module 830 may display data and/or information associated with or generated by the imaging system 100. The display module 830 may be the same as or similar to the display module 730 as described elsewhere in the present disclosure (e.g., FIG. 7 and descriptions thereof).

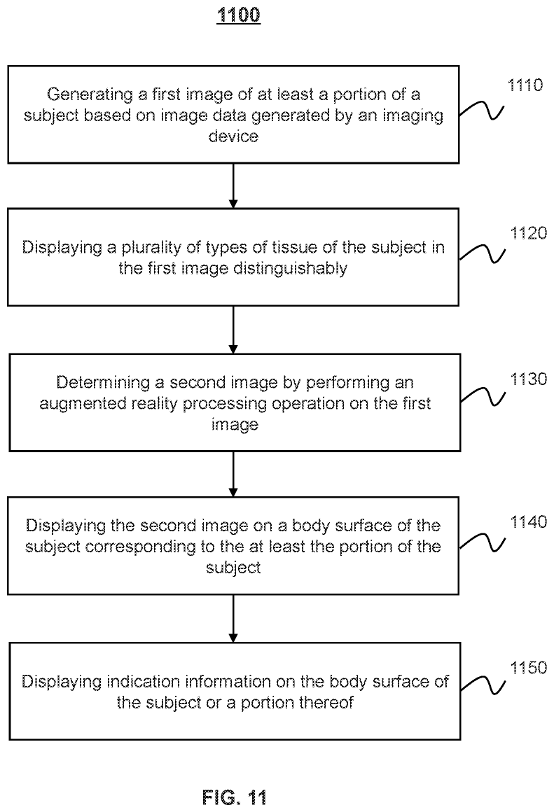

The processing module 840 may be configured to cause a medical operation to be performed on a portion of a subject. In some embodiments, the processing module 840 may be operably connected to an arm and one or more operating elements of the arm. The processing module 840 may cause the arm and the one or more operating elements of the arm to perform the medical operation on the portion of the subject based on a first image of the portion of the subject, a second image displayed on a body surface of the subject corresponding to the portion of the subject, and/or indication information displayed on the body surface of the subject. More descriptions of the medical operation may be found elsewhere in the present disclosure (e.g., FIG. 11, and descriptions thereof).

In some embodiments, one or more modules illustrated in FIG. 8 may be implemented in at least part of the imaging system 100 as illustrated in FIG. 1. For example, the image generation module 810, the augmented reality module 820, the display module 830, and the processing module 840 may be integrated into a console (not shown). Via the console, a user may set parameters for implementing operations described elsewhere in the present disclosure. In some embodiments, the modules described in FIG. 8 may be implemented via the processing device 120 and/or the terminal 140.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be added or omitted in the processing device 120. For example, the processing device 120 may further include a storage module (not shown in FIG. 8) configured to store data and/or information (e.g., a first image, a second image) associated with the imaging system 100. As another example, the processing module 840 may be omitted. The medical operation may be performed by a user of the imaging system 100.

FIG. 9 is a flowchart illustrating an exemplary process for displaying a second image on a body surface of a subject according to some embodiments of the present disclosure. In some embodiments, the process 900 may be implemented in the imaging system 100 illustrated in FIG. 1. For example, the process 900 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting.

In 910, the processing device 120 (e.g., the image generation module 710, the image generation module 810) may generate a first image of a portion of a subject based on image data generated by an medical device (e.g., the medical device 110).

In some embodiments, the medical device (e.g., the medical device 110) may be a computed tomography (CT) device, a magnetic resonance imaging (MRI) device, a positron emission tomography (PET) device, an ultrasound device, an X-ray imaging device, a digital subtraction angiography (DSA) device, a dynamic spatial reconstruction (DSR) device, a multimodality device (e.g., a PET-CT device, a CT-MRI device), or the like, as described elsewhere in the present disclosure. In some embodiments, the medical device may be a C-arm device. The C-arm device may perform a real time cone beam CT scan on the portion of the subject during a medical operation. A real time image reflecting a real time status of the subject may be determined based on the real time cone beam CT scan. Therefore, an inconsistency in a position of the subject and/or a body status of the subject at the time of imaging and the medical operation may be avoided or reduced. The accuracy of the first image may be improved, which may facilitate a subsequent augmented reality processing, diagnosis, and/or image-guided operation performed manually, semi-automatically, or fully automatically.

In some embodiments, the processing device 120 may reconstruct the image data to generate the first image based on one or more reconstruction techniques. Exemplary reconstruction techniques may include an iterative reconstruction algorithm, e.g., a maximum likelihood expectation maximization (MLEM) algorithm, an ordered subset expectation maximization (OSEM) algorithm, a maximum-likelihood reconstruction of attenuation and activity (MLAA) algorithm, a maximum-likelihood attenuation correction factor (MLACF) algorithm, a maximum likelihood transmission reconstruction (MLTR) algorithm, a conjugate gradient algorithm, a maximum-a-posteriori estimation algorithm, a filtered back projection (FBP) algorithm, a 3D reconstruction algorithm, or the like, or any combination thereof.

In some embodiments, the first image may be a CT image, an MRI image, a PET image, an ultrasound image, an X-ray image, a DSA image, a DSR image, a multimodality image (e.g., a PET-CT image, a CT-MRI image), or the like, or any combination thereof. In some embodiments, the first image may be a two-dimensional image, a three-dimensional image, or the like, or a combination thereof.

In some embodiments, the first image may include information associated with the portion of the subject. In some embodiments, the portion of the subject may include one or more region(s) of interest of the subject that need to be processed (or treated) by a medical instrument during a medical operation. In some embodiments, the region(s) of interest may include an organ (e.g., a lung, the liver, the heart, etc.) or a portion thereof (e.g., a tumor, a nodule, a bleeding spot, or the like, or any combination thereof). In some embodiments, the region(s) of interest may be determined based on one or more image segmentation algorithms. Exemplary image segmentation algorithms may include a threshold segmentation algorithm, a region growing algorithm, a watershed segmentation algorithm, a morphological segmentation algorithm, a statistics segmentation algorithm, or the like, or any combination thereof. In some embodiments, the first image may include information associated with a plurality of types of tissue of the subject and/or information associated with a plurality of organs of the subject. The plurality of types of tissue of the subject may include a muscle tissue, a connective tissue, an epithelial tissue, a nervous tissue, or the like. Exemplary information associated with the plurality of types of tissue of the subject may include relative positions of the plurality of types of tissue inside the subject, shapes of the plurality of types of tissue, sizes of the plurality of types of tissue, or the like, or any combination thereof. The plurality of organs of the subject may include the liver, the stomach, the heart, the lungs, or the like. Exemplary information associated with the plurality of organs of the subject may include relative positions of the plurality of organs inside the subject, shapes of the plurality of organs, sizes of the plurality of organs, or the like, or any combination thereof, represented in the first image.

In 920, the processing device 120 (e.g., the augmented reality module 720, the augmented reality module 820) may determine a second image by performing an augmented reality processing operation on the first image.

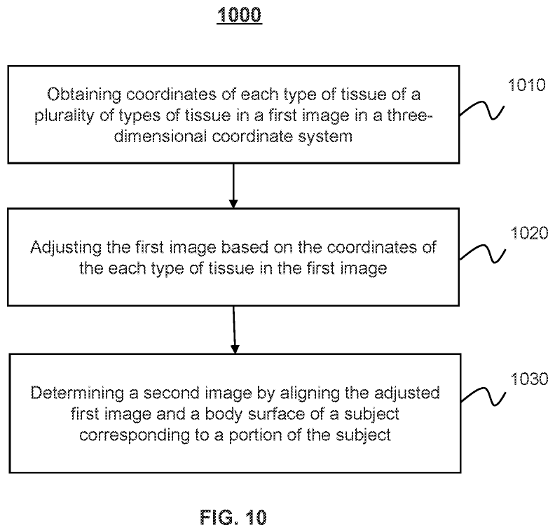

In some embodiment, the processing device 120 may perform the augmented reality processing operation on the first image according to an augmented reality technology. As used herein, the augmented reality (AR) may refer to an interactive experience of a real-world environment where objects that reside in the real-world are enhanced by computer-generated perceptual information, sometimes across multiple sensory modalities, including visual, auditory, haptic, somatosensory and olfactory. More descriptions of the determination of the second image may be found elsewhere in the present disclosure (e.g., FIG. 10, and descriptions thereof).

In some embodiments, the second image may be an AR image. In some embodiments, the AR image may present a scene of the subject (or a portion thereof), the one or more regions of interest, and/or a scene of one or more medical instruments, or the like. In some embodiments, the AR image may be a 3D dynamic AR image or a 3D AR video of the object. In some embodiments, the second image may include information associated with the plurality of types of tissue of the subject and/or information associated with the plurality of organs of the subject. In some embodiments, the information associated with the plurality of types of tissue of the subject and/or the information associated with the plurality of organs of the subject represented in the second image may be determined based on the information associated with the plurality of types of tissue of the subject and/or the information associated with the plurality of organs of the subject in the subject.

In 930, the processing device 120 (e.g., the display module 730, the display module 830) or an interaction device (e.g., the interaction device 160) may display the second image on a body surface of the subject corresponding to the portion of the subject.

In some embodiments, the display module 730 may include a video display (e.g., an electroluminescent display, an electronic paper, a light-emitting diode (LED) display, a liquid crystal display (LCD), a plasma display, a digital micromirror device (DMD), a liquid on silicon display, a field emission display, a laser color video display, a quantum dot display, an interferometric modulator display, a flexible display, etc.), a non-video display (e.g., a vacuum fluorescent display, a seven segment display, etc.), a 3D display (e.g., a holographic display, a retina display, a fog display, etc.), or the like, or a combination thereof. An exemplary display may be a head mounted display (HMD), a display device (e.g., a flat panel display or a curved panel display), or the like.

In some embodiments, the interaction device (e.g., the interaction device 160) may include an optical device (e.g., an augmented reality device), a projection device (e.g., the projection device 430), or the like, or any combination thereof, as described elsewhere in the present disclosure. For example, the user may view the second image displayed on the body surface of the subject corresponding to the portion of the subject by wearing AR glasses or an AR helmet. Accordingly, by allowing the user to view the second image via AR glasses or an AR helmet, the second image does not need to be projected in a space, thereby saving the need for a projection device or a space where the second image is projected. As another example, the projection device (e.g., the projection device 430) may project the second image on the body surface of the subject corresponding to the portion of the subject. Accordingly, the user may view the second image displayed on the body surface of the subject corresponding to the portion of the subject at various positions and angles directly, so that the user may perform a subsequent medical operation (e.g., an incision operation, a puncture operation) according to the second image. In addition, the projected second image may be stable, and avoid or reduce an adverse effect such as visual fatigue.

In some embodiments, the user may adjust a display effect of the second image (e.g., an AR image). For example, the user may choose to zoom in or out, and/or drag the AR image so that the user may view different portions (or scopes) of the subject with different amplifications. The processing device 120 may receive instruction(s) associated with the display effect of the AR image from the user and perform the corresponding zooming in or out and/or dragging operations, or the like, to realize the desired display effect.

According to some embodiments of the present disclosure, the user may simultaneously view the second image (e.g., an AR image) associated with the portion of the subject, and an interior structure of the subject, or a portion thereof, displayed on the body surface of the subject, and a surrounding environment (e.g., one or more medical instruments) of the portion of the subject. Therefore, a medical operation may be performed conveniently by the user with the guidance of the second image (e.g., an AR image), which may reduce the use of the imaging device and accordingly reduce the exposure of the user to harmful radiation during the medical operation.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a medical operation may be added after operation 930 as described elsewhere in the present disclosure (e.g., FIG. 11, and descriptions thereof).

FIG. 10 is a flowchart illustrating an exemplary process for determining a second image according to some embodiments of the present disclosure. In some embodiments, the process 1000 may be implemented in the imaging system 100 illustrated in FIG. 1. For example, the process 1000 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting.

In 1010, the processing device 120 (e.g., the augmented reality module 720, the augmented reality module 820) may obtain coordinates of each type of tissue of a plurality of types of tissue in a first image in a three-dimensional coordinate system.

In some embodiments, the processing device 120 may determine the coordinates of the each type of tissue of the plurality of types of tissue (and/or coordinates of each organ of a plurality organs) in the first image (e.g., a three-dimensional image) in the three-dimensional coordinate system according to a marker corresponding to the each type of tissue of the plurality of types of tissue (and/or a marker corresponding to the each organ of the plurality of organs). For example, the processing device 120 may determine a specific position of tissue of interest as the marker corresponding to the tissue. The specific position of the tissue may not be easily displaced or deformed, and may be easily distinguished. As another example, the marker may be installed on a table (e.g., the table 118a, the table 118b) that supports the subject.

In some embodiments, the processing device 120 may determine coordinates of the marker in a real world coordinate system. The processing device 120 may then determine coordinates of the marker in the first image in the three-dimensional coordinate system (also referred to as an image coordinate system). The processing device 120 may further determine the coordinates of the each type of tissue of the plurality of types of tissue in the first image in the three-dimensional coordinate system based on the coordinates of the marker in the first image in the three-dimensional coordinate system and the coordinates of the marker in the real world coordinate system. Accordingly, the processing device 120 may determine relative positions of the plurality of types of tissue, shapes of the plurality of types of tissue, and sizes of the plurality of types of tissue, in the first image. As used herein, the real world coordinate system refers to a fixed coordinate system for representing an object in the real world. The image coordinate system refers to a coordinate system that describes positions of an object in an image.

In 1020, the processing device 120 (e.g., the augmented reality module 720, the augmented reality module 820) may adjust the first image based on the coordinates of the each type of tissue in the first image.

In some embodiments, during a medical operation, a position of a user and/or a position of an interaction device (e.g., an AR device) on the user may change. The processing device 120 may adjust the first image based on the position of the user and/or the position of the interaction device (e.g., an AR device). For example, the processing device 120 may adjust the relative positions of the plurality of types of tissue, the shapes of the plurality of types of tissue, and the sizes of the plurality of types of tissue in the first image based on relative positions of the user (or the interaction device) and the marker corresponding to the each type of tissue of the plurality of types of tissue. Accordingly, the processing device 120 may determine the adjusted first image associated with the portion of the subject corresponding to different viewing angles of the user at different positions.

In 1030, the processing device 120 (e.g., the augmented reality module 720, the augmented reality module 820) may determine a second image by aligning the adjusted first image and a body surface of a subject corresponding to a portion of the subject.

In some embodiments, the interaction device may be a projection device. The processing device 120 may align the adjusted first image and the body surface of the subject corresponding to the portion of the subject based on coordinates of the body surface of the subject in the real world coordinate system and coordinates of the each type of tissue of the plurality of types of tissue in the first image in the three dimensional coordinate system. The processing device 120 may convert the coordinates of the each type of tissue of the plurality of types of tissue in the first image from the three-dimensional coordinate system into the real-world coordinate system. The processing device 120 may align the adjusted first image on the body surface of the subject corresponding to the portion of the subject based on the coordinates of the body surface of the subject in the real world coordinate system and the coordinates of the each type of tissue of the plurality of types of tissue in the first image in the real world coordinate system. In some embodiments, the processing device 120 may align the adjusted first image and the body surface of the subject corresponding to the portion of the subject by adjusting one or more parameters of the projection device. The one or more parameters of the projection device may include a focal length of the lens of the projection device, a projection angle, or the Ike, or any combination thereof.

In some embodiments, the interaction device may be an optical device (e.g., AR glasses). The processing device 120 may align the adjusted first image and the body surface of the subject corresponding to the portion of the subject according to an object detection algorithm. Exemplary object detection algorithms may include an inter-frame difference algorithm, a background difference algorithm, an optical flow algorithm, or the like, or any combination thereof. For example, the processing device 120 may identify one or more regions on the body surface of the subject according to the object detection algorithm. The processing device 120 may display the adjusted first image on a screen (e.g., a transparent screen through which the subject or a portion thereof is visible) of the optical device by aligning the detected one or more regions of the subject and one or more corresponding portions of the adjusted first image.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted.

FIG. 11 is a flowchart illustrating an exemplary process for displaying a second image on a body surface of a subject according to some embodiments of the present disclosure. In some embodiments, the process 1100 may be implemented in the imaging system 100 illustrated in FIG. 1. For example, the process 1100 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1100 may be accomplished with one or more additional operations not described, and/or without one or more of the operations discussed. Additionally, the order in which the operations of the process 1100 as illustrated in FIG. 11 and described below is not intended to be limiting.

In 1110, the processing device 120 (e.g., the image generation module 710, the image generation module 810) may generate a first image of a portion of a subject based on image data generated by a medical device. More descriptions of the generation of the first image may be found elsewhere in the present disclosure (e.g., operation 910 in FIG. 9, and descriptions thereof).

In 1120, the processing device 120 (e.g., the display module 730, the display module 830) or an interaction device (e.g., the interaction device 160) may display a plurality of types of tissue of the subject in the first image distinguishably.

In some embodiments, the processing device 120 may display the plurality of types of tissue (and/or a plurality of organs) of the subject in the first image distinguishably by performing a rendering operation (e.g., a three-dimensional (3D) rendering operation) on the first image. As used herein, a 3D rendering may refer to a 3D computer graphics process of converting 3D wire frame models into 2D images on a computer. For example, the processing device 120 may render the plurality of types of tissue (and/or the plurality of organs) of the subject in the first image, such that the user may visually distinguish the plurality of types of tissue (and/or the plurality of organs) of the subject in a processed first image (e.g., a second image). Accordingly, overlapping of the plurality of types of tissue (and/or the plurality of organs) of the subject in the first image may also be avoided.

In some embodiments, the processing device 120 may display the plurality of types of tissue (and/or the plurality of organs) of the subject in the first image in different colors, different grayscales, and/or different textures. For example, the plurality of types of tissue (and/or the plurality of organs) of the subject may be presented by different contrasting colors. In some embodiments, the processing device 120 may display a specific organ (e.g., the heart) of the subject in a conspicuous color (e.g., red, yellow).

In 1130, the processing device 120 (e.g., the augmented reality module 720, the augmented reality module 820) may determine a second image by performing an augmented reality processing operation on the first image. More descriptions of the determination of the second image may be found elsewhere in the present disclosure (e.g., operation 920 in FIG. 9, FIG. 10, and descriptions thereof).

In 1140, the processing device 120 (e.g., the display module 730, the display module 830) or an interaction device (e.g., the interaction device 160) may display the second image on a body surface of the subject corresponding to the portion of the subject. More descriptions of the display of the second image may be found elsewhere in the present disclosure (e.g., operation 930 in FIG. 9, and descriptions thereof).

In 1150, the processing device 120 (e.g., the display module 730, the display module 830) or an interaction device (e.g., the interaction device 160) may display indication information on the body surface of the subject or a portion thereof.

In some embodiments, the indication information may include a preset slit position, a preset needle insertion direction, a preset puncture path, a preset puncture needle position, or the like, or any combination thereof. In some embodiments, the indication information may be manually set by the user of the imaging system 100 or be determined by one or more components (e.g., the processing device 120) of the imaging system 100 based on the first image of the portion of the subject.

For illustration purposes, if a medical operation targets a lung nodule, the processing device 120 may identify a target region of the subject in which the lung nodule is located by segmenting the first image or the second image generated based on the first image (e.g., an AR image). The processing device 120 may determine a target position (e.g., a preset slit position, a preset puncture needle position) in the subject for operating the medical instrument based on the position of segmented target region of the subject in the AR image. For example, the processing device 120 may determine a center position of the lung nodule as the target position. The processing device 120 may display information relating to the target region and the target position on the body surface of the subject, and guide the operation by the user.

Accordingly, the medical operation may be performed conveniently with the guidance of the second image and the indication information displayed on the body surface of the subject, and accordingly the efficiency and/or the accuracy of the medical operation may be improved, which may reduce trauma to the subject from the operation and/or avoid excessive imaging accompanying the operation.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more operations may be added or omitted. For example, a medical operation may be added after operation 1150. The medical operation may include an incision operation, a puncture operation, a surgery, or the like, or a combination thereof. In some embodiments, the medical operation may be performed by the user of the imaging system 100. In some embodiments, the processing device 120 (e.g., the processing module 840) may cause the medical operation to be performed on the portion of the subject via one or more arms and one or more operating elements of each arm of the one or more arms. In some embodiments, the processing device 120 may be operably connected to the arm and the one or more operating elements of the arm. For example, the arm may be a robotic arm. The one or more operating elements may be connected to an end of the arm. In some embodiments, the type of the arm or the operating element and the number (or count) of the arm or the operating element may be determined based on an actual need of the medical operation. The operating element may include a plurality of medical instruments to achieve a variety of medical processing functions. For example, the operating element may include a scalpel, a puncture needle, or the like.

In some embodiments, the arm and the one or more operating elements of the arm may be manually controlled by the user or be automatically controlled by one or more components (e.g., the processing device 120) of the imaging system 100. For example, the processing device 120 may transmit a control signal to the arm and the one or more operating elements of the arm to cause the medical operation to be performed on the portion of the subject based on the second image and the indication information (e.g., the preset slit position, the preset needle insertion direction, the preset puncture path, the preset puncture needle position). The arm and the one or more operating elements may perform the medical operation on the portion of the subject based on the control signal.

In some embodiments, the medical operation may be monitored in real time based on the indication information displayed on the body surface of the subject. For example, during the medical operation, the processing device 120 may obtain an actual puncture needle position via a sensor installed in a puncture needle in real time. The processing device 120 may determine whether the puncture needle is puncture according to the preset puncture path by comparing the preset puncture needle position and the actual puncture needle position. In response to a determination that the puncture needle is not puncture according to the preset puncture path, the processing device 120 may generate a reminder to inform the user to adjust the puncture needle position or stop puncturing. Accordingly, the safety of the medical operation may be improved.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the users computer, partly on the users computer, as a stand-alone software package, partly on the user's computer and partly on a remote computer or entirely on the remote computer or server. In the latter scenario, the remote computer may be connected to the users computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

1. A system, comprising:
an imaging device configured to generate image data by imaging a subject or a portion thereof;
a storage device configured to store information regarding a plurality of preset postures; and
a processing device configured to communicate with the imaging device, the storage device, and an interaction device, wherein
the interaction device, in communication with the storage device, is configured to provide to the subject information regarding at least one preset posture of the plurality of preset postures.

2. The system of claim 1, wherein the interaction device includes at least one of an optical device, a projection device, or an audio device.

3. The system of claim 2, wherein the interaction device includes a holographic projector configured to project a first image of the at least one preset posture of the plurality of preset postures.

4. The system of claim 3, wherein the holographic projector is movable.

5. The system of claim 4, further including a movable base configured to carry the holographic projector to move.

6. (canceled)

7. The system of claim 1, wherein the processing device is further configured to determine a display position of the information regarding the at least one preset posture based on a position of the imaging device.

8. The system of claim 1, further comprising:
an image capture device configured to capture a second image representing an actual posture of the subject when the subject is positioned within the imaging device.

9. The system of claim 8, wherein the processing device is further configured to:
determine a difference between the actual posture of the subject and the at least one preset posture of the subject; and
determine whether the difference is below a threshold.

10. The system of claim 9, wherein the processing device is further configured to:
generate a reminder in response to a determination that the difference exceeds the threshold.

11-12. (canceled)

13. The system of claim 1, wherein
the processing device is further configured to
generate a first image of the portion of the subject based on the image data generated by the imaging device; and
determine a second image by performing an augmented reality processing operation on the first image, and
the interaction device is configured to display the second image on a body surface of the subject corresponding to the portion of the subject.

14. The system of claim 13, wherein the processing device is further configured to cause a medical operation to be performed on the portion of the subject.

15. The system of claim 14, wherein the processing device is operably connected to an arm.

16. The system of claim 15, wherein
the processing device is operably connected to at least one operating element of the arm, and
the at least one operating element includes at least one of a scalpel or a puncture needle.

17-19. (canceled)

20. A system, comprising:
an imaging device configured to generate image data by imaging a subject or a portion thereof; and
a processing device configured to
communicate with the imaging device and an interaction device,
generate a first image of the portion of the subject based on the image data generated by the imaging device; and
determine a second image by performing an augmented reality processing operation on the first image, and
the interaction device is configured to display the second image on a body surface of the subject corresponding to the portion of the subject.

21-38. (canceled)

39. A method implemented on a computing device having one or more processors and one or more storage devices, the method comprising:
generating a first image of at least a portion of a subject based on image data generated by an imaging device;
determining a second image by performing an augmented reality processing operation on the first image; and
displaying the second image on a body surface of the subject corresponding to the at least the portion of the subject.

40. The method of claim 39, wherein the first image includes information associated with a plurality of types of tissue of the subject, and the method further comprises:
displaying the plurality of types of tissue of the subject in the first image distinguishably.

41. The method of claim 40, wherein displaying the plurality of types of tissue of the subject in the first image distinguishably comprises:
displaying the plurality of types of tissue of the subject in the first image in different colors, different grayscales, or different textures.

42. The method of claim 41, wherein the first image is a three-dimensional image, and determining a second image by performing an augmented reality processing on the first image comprises:
obtaining coordinates of each type of tissue of the plurality of types of tissue in the first image in a three-dimensional coordinate system;
adjusting the first image based on the coordinates of the each type of tissue in the first image; and
determining the second image by aligning the adjusted first image and the body surface of the subject corresponding to the portion of the subject.

43. The method of claim 39, further comprising:
displaying indication information on the body surface of the subject.

44. The method of claim 43, wherein the indication information includes at least one of a preset slit position, a preset needle insertion direction, a preset puncture path, or a preset puncture needle position.

45-63. (canceled)
